SEQUENCE LISTING (16 sequences)

SEQ ID NO: 1 - DNA, 42 bp, synthetic construct
  misc_feature 1..42: note = [Primary] anti-HER2 aptamer that may be used in the dual-specific aptamers set forth in SEQ ID NOs: 8 to 14 (i.e., dsA1 to dsA10)
  source 1..42: mol_type = other DNA
gcagcggtgtgggggcagcggtgtgggggcagcggtgtgggg

SEQ ID NO: 2 - DNA, 34 bp, synthetic construct
  misc_feature 1..34: note = [Primary] anti-CD16 patamer; A truncated form of the anti-CD16 aptamer set forth in SEQ ID NO: 3; May be used in the dual-specific aptamers set forth in SEQ ID NOs: 8, 10, 12 and 13 (i.e., dsA1, 3, 5, 6, 8 and 9).
  source 1..34: mol_type = other DNA
ccactgcgggggtctatacgtgaggaagaagtgg

SEQ ID NO: 3 - DNA, 41 bp, synthetic construct
  misc_feature 1..41: note = [primary] anti-CD16 aptamer that may be used in the dual-specific aptamers set forth in SEQ ID NOs: 9, 11 and 14 (i.e., dsA2, 4, 7 and 10).
  source 1..41: mol_type = other DNA
ccactgcgggggtctatacgtgaggaagaagtgggcaggtc

SEQ ID NO: 4 - skipped

SEQ ID NO: 5 - DNA, 13 bp, synthetic construct
  misc_feature 1..13: note = Nucleic acid linker used to attach 5' end of the anti-HER2 segment to 3' end of the anti-CD16 segment of the dual-specific aptamer set forth in SEQ ID NO: 11 (i.e., dsA4).
  source 1..13: mol_type = other DNA
aaaaaaaaaaaaa

SEQ ID NO: 6 - DNA, 20 bp, synthetic construct
  misc_feature 1..20: note = Nucleic acid linker used to attach one end of the anti-HER2 segment to one end of the anti-CD16 segment at the dual-specific aptamers set forth in SEQ ID NOs: 12, 13 and 14 (i.e., dsA5 to 10).
  source 1..20: mol_type = other DNA
aaaaaaaaaaaaaaaaaaaa

SEQ ID NO: 7 - DNA, 20 bp, synthetic construct
  misc_feature 1..20: note = Poly-Thymidine sequence that may be hybridized to the nucleic acid linker of the dual-specifc aptamers set forth in SEQ ID NOs: 12 to 14 (dsA8, 9 and 10), and may be a complementary sequence of the nucleic acid linker set forth in SEQ ID NO: 6.
  source 1..20: mol_type = other DNA
tttttttttttttttttttt

SEQ ID NO: 8 - DNA, 76 bp, synthetic construct
  misc_feature 1..76: note = Dual-specific aptamer (1) referred to as dsA1 throughout the disclosure, wherein the anti-HER2 aptamer (SEQ ID NO: 1) may be disposed at 3' arm and the anti-CD16 aptamer (SEQ ID NO: 2) may be disposed at 5' arm thereof.
  source 1..76: mol_type = other DNA
ccactgcgggggtctatacgtgaggaagaagtgggcagcggtgtgggggcagcggtgtgggggcagcggtgtgggg

SEQ ID NO: 9 - DNA, 83 bp, synthetic construct
  misc_feature 1..83: note = Dual-specific aptamer (2) referred to as dsA2 throughout the disclosure, wherein the anti-HER2 aptamer (SEQ ID NO: 1) may be disposed at 3' arm and the anti-CD16 aptamer (SEQ ID NO: 3) may be disposed at 5' arm thereof.
  source 1..83: mol_type = other DNA
ccactgcgggggtctatacgtgaggaagaagtgggcaggtcgcagcggtgtgggggcagcggtgtgggggcagcggtgtgggg

SEQ ID NO: 10 - DNA, 83 bp, synthetic construct
  misc_feature 1..83: note = Dual-specific aptamer (3) referred to as dsA3 throughout the disclosure, wherein the anti-HER2 aptamer (SEQ ID NO: 1) may be disposed at 3' arm and the anti-CD16 aptamer (SEQ ID NO: 2) may be disposed at 5' arm thereof.
  source 1..83: mol_type = other DNA
ccactgcgggggtctatacgtgaggaagaagtggaaaaaaagcagcggtgtgggggcagcggtgtgggggcagcggtgtgggg

SEQ ID NO: 11 - DNA, 96 bp, synthetic construct
  misc_feature 1..96: note = Dual-specific aptamer (4) referred to as dsA4 throughout the disclosure, wherein the anti-HER2 aptamer (SEQ ID NO: 1) may be disposed at 3' arm and the anti-CD16 aptamer (SEQ ID NO: 3) may be disposed at 5' arm thereof.
  source 1..96: mol_type = other DNA
ccactgcgggggtctatacgtgaggaagaagtgggcaggtcaaaaaaaaaaaaagcagcggtgtgggggcagcggtgtgggggcagcggtgtgggg

SEQ ID NO: 12 - DNA, 96 bp, synthetic construct
  misc_feature 1..96: note = Dual-specific aptamers referred to as dsA5 and dsA8, wherein the anti-HER2 (SEQ ID NO: 1) aptamer may be disposed at 3' arm and the anti-CD16 aptamer (SEQ ID NO: 2) may be disposed at 5' arrm thereof; dsA8 may have a double-stranded linker.
  source 1..96: mol_type = other DNA
ccactgcgggggtctatacgtgaggaagaagtggaaaaaaaaaaaaaaaaaaaagcagcggtgtgggggcagcggtgtgggggcagcggtgtgggg

SEQ ID NO: 13 - DNA, 96 bp, synthetic construct
  misc_feature 1..96: note = Dual-specific aptamers referred to as dsA6 and dsA9, wherein the anti-HER2 aptamer (SEQ ID NO: 1) may be disposed at 5' arm and the anti-CD16 aptamer (SEQ ID NO: 2) may be disposed at 3' arrm thereof; dsA9 may have a double-stranded linker.
  source 1..96: mol_type = other DNA
gcagcggtgtgggggcagcggtgtgggggcagcggtgtggggaaaaaaaaaaaaaaaaaaaaccactgcgggggtctatacgtgaggaagaagtgg

SEQ ID NO: 14 - DNA, 103 bp, synthetic construct
  misc_feature 1..103: note = Dual-specific aptamers referred to as dsA7 and dsA10, wherein the anti-HER2 aptamer (SEQ ID NO: 1) may be disposed at 5' arm and the anti-CD16 aptamer (SEQ ID NO: 3) may be disposed at 3' arrm thereof; dsA9 may have a double-stranded linker.
  source 1..103: mol_type = other DNA
gcagcggtgtgggggcagcggtgtgggggcagcggtgtggggaaaaaaaaaaaaaaaaaaaaccactgcgggggtctatacgtgaggaagaagtgggcaggtc

SEQ ID NO: 15 - DNA, 86 bp, synthetic construct
  misc_feature 1..86: note = [Primary] anti-HER2 aptamer as a candidate to be incorporated in the dual-specific aptamers.
  source 1..86: mol_type = other DNA
aaccgcccaaatccctaagagtctgcacttgtcattttgtatatgtatttggtttttggctctcacagacacactacacacgcaca

SEQ ID NO: 16 - DNA, 40 bp, synthetic construct
  misc_feature 1..40: note = [Primary] anti-HER2 aptamer as a candidate to be incorporated in the dual-specific aptamers.
  source 1..40: mol_type = other DNA
tctaaaaggattcttcccaaggggatccaattcaaacagc

(19) United States
(12) Patent Application Publication
(10) Pub. No.: US 2024/0060073 A1
(43) Pub. Date: Feb. 22, 2024

(54) DUAL-SPECIFIC APTAMER TRIGGERING CELL-MEDIATED CYTOTOXICITY TO LYSE HER2-POSITIVE CANCER CELLS

(71) Applicants: Majid Talkhabifard, Gorgan (IR); Majid Shahbazi, Gorgan (IR)

(72) Inventors: Majid Talkhabifard, Gorgan (IR); Majid Shahbazi, Gorgan (IR)

(73) Assignees: Majid Talkhabifard, Gorgan (IR); Majid Shahbazi, Gorgan (IR); Aria Tina Gene Company, Gorgan (IR)

(21) Appl. No.: 18/468,726

(22) Filed: Sep. 17, 2023

(51) Int. Cl. C12N 15/115; C07K 16/28; C07K 16/32

(52) U.S. Cl. CPC C12N 15/115; C07K 16/283; C07K 16/32; C07K 2317/31; C07K 2317/73; C12N 2310/16

(57) ABSTRACT

A dual-specific aptamer comprising the nucleic acid sequence set forth in SEQ ID NO: 14. The dual-specific aptamer comprising an anti-HER2 segment capable of binding to a HER2 marker and an anti-CD16 segment capable of binding to a CD16 marker. The anti-HER2 segment comprises nucleotide 1 to nucleotide 42 of SEQ ID NO: 14, and the anti-CD16 segment comprises nucleotide 63 to nucleotide 103 of SEQ ID NO: 14. The dual-specific aptamer further comprises a nucleic acid linker comprising nucleotide 43 to nucleotide 62 of SEQ ID NO: 14 and is selected from the group consisting of a double-stranded nucleic acid linker or a single-stranded nucleic acid linker.

### CROSS-REFERENCE TO RELATED APPLICATION

This application is a continuation-in-part of PCT Application PCT/2021/062450, filed on Dec. 30, 2021, entitled “DUAL-SPECIFIC APTAMER TRIGGERING CELL-MEDIATED CYTOTOXICITY TO LYSE HER2-POSITIVE CANCER CELLS,” which is incorporated herein by reference in their entirety.

### TECHNICAL FIELD

The present disclosure generally relates to an exemplary dual-specific aptamer, and more particularly to an exemplary dual-specific aptamer that may include an exemplary anti-HER2 segment configured to specifically bind to a HER2 marker and an exemplary anti-CD16 segment configured to specifically bind to a CD16 marker.

### BACKGROUND

Nucleic acid aptamers are small oligonucleotides capable of forming different three-dimensional structures; these oligonucleotides have shown binding affinity to a wide range of target molecules via non-covalent interactions. Aptamers have demonstrated remarkable advantages in therapeutic and diagnostic applications, such as high affinity, excellent specificity, low immunogenicity, simple synthesis, and simple modification; these properties have made aptamers promising candidates for use in various diagnostic and therapeutic applications. In recent years, aptamers have gained increasing attention as modern therapeutic agents in oncology studies. Main strategies of aptamer-based therapeutics may include blockade of receptor-ligand interactions through antagonistic activity of aptamer, and targeted drug delivery.

Human epidermal growth factor receptor 2 (Her2/neu) is one of the four members of epidermal growth factor receptor (EGFR) family involved in growth, proliferation, and differentiation of cells. Heterodimerization of HER2 with any of the three members of the EGFR family may result in autophosphorylation of tyrosine residues inside the receptors' cytoplasmic domain and activation of a variety of signaling pathways. Over-expression of HER2 may play an essential role in the development and progression of different cancers, such as breast cancer, gastric/gastroesophageal cancers, etc. HER2 may be a suitable target for anticancer-therapy of tumors having an over-expression of HER2/neu receptor. A majority of HER2-targeted therapies for treating HER2-positive breast cancers are based on tyrosine kinase inhibitors (e.g., neratinib and lapatinib) and monoclonal antibodies (mAbs; e.g., Trastuzumab and pertuzumab) that may lead to pathological signaling inhibition or activation of immune system. The anti-HER2 monoclonal antibodies, such as Trastuzumab, may block HER2 by preventing dimerization; they may also activate antibody-dependent cell-mediated cytotoxicity (ADCC) mechanism against HER2-positive cancer cells by binding, through their Fc-region, to Natural Killer (NK) cells expressing FcγRIIIA/CD16. Nevertheless, HER2-targeted therapy using monoclonal antibodies may be associated with severe side effects that limit their administration in some cases.

Thereby, there is need to produce therapeutically-effective aptamers, as alternatives to monoclonal antibodies, for treating HER2-overexpressing (HER2-positive) cancers.

### SUMMARY

This summary is intended to provide an overview of the subject matter of one or more exemplary embodiments, and is not intended to identify essential elements or key elements of the subject matter, nor is it intended to be used to determine the scope of the claimed implementations. Its sole purpose is to present some concepts of one or more aspects in a simplified form as a prelude to the more detailed description that is presented later. The proper scope of one or more exemplary embodiments may be ascertained from the claims set forth below in view of the detailed description below and the drawings.

One or more exemplary embodiments describe an exemplary dual-specific aptamer comprising an exemplary nucleic acid sequence set forth in SEQ ID NO: 14. In one or more exemplary embodiments, an exemplary dual-specific aptamer may comprise an exemplary anti-HER2 segment capable of binding to a HER2 marker and an exemplary anti-CD16 segment capable of binding to a CD16 marker. In an exemplary embodiment, an exemplary anti-HER2 segment may comprise nucleotide 1 to nucleotide 42 of SEQ ID NO: 14 and an exemplary anti-CD16 segment may comprise nucleotide 63 to nucleotide 103 of SEQ ID NO: 14.

In one or more exemplary embodiments, an exemplary dual-specific aptamer may comprise an exemplary double-stranded nucleic acid linker configured to attach the 3′ end of an exemplary anti-HER2 segment to the 5′ end of an exemplary anti-CD16 segment. An exemplary double-stranded nucleic acid linker may comprise an exemplary poly-Adenosine (A) sequence hybridized to a poly-thymidine (T) sequence. In an exemplary embodiment, an exemplary poly-A sequence may include nucleotide 43 to nucleotide 62 of SEQ ID NO: 14.

In one or more exemplary embodiments, an exemplary dual-specific aptamer may comprise an exemplary single-stranded nucleic acid linker configured to attach the 3′ end of an exemplary anti-HER2 segment to the 5′ end of an exemplary anti-CD16 segment. An exemplary single-stranded nucleic acid linker may comprise a poly-A sequence comprising nucleotide 43 to nucleotide 62 of SEQ ID NO: 14.

This Summary may introduce a number of concepts in a simplified format; the concepts are further disclosed within the “Detailed Description” section. This Summary is not intended to configure essential/key features of the claimed subject matter, nor is intended to limit the scope of the claimed subject matter.

### BRIEF DESCRIPTION OF THE DRAWINGS

The novel features which are believed to be characteristic of the present disclosure, as to its structure, organization, use and method of operation, together with further objectives and advantages thereof, will be better understood from the following drawings in which an exemplary embodiment will now be illustrated by way of example. It is expressly understood, however, that the drawings are for the purpose of illustration and description only and are not intended as a definition of the limits of the present disclosure. Exemplary embodiments will now be described by way of example in association with the accompanying drawings in which:

FIG. 1 illustrates predicted secondary structures of exemplary dual-specific aptamers (dsA1 to 10) and their corresponding flow cytometry binding analysis after incubation with PBMCs (peripheral blood mononuclear cell) and enriched NK cells, consistent with one or more exemplary embodiments of the present disclosure;

FIG. 2 illustrates predicted secondary structures of exemplary dual-specific aptamers (dsA1 to 10) and their corresponding flow cytometry binding analysis after incubation with SKBR3 cells as HER2-positive breast cancer cells, consistent with one or more exemplary embodiments of the present disclosure;

FIG. 3A illustrates a bar chart representing cytotoxic effect of an exemplary dsA7 (SEQ ID NO: 14) and an exemplary dsA10 (SEQ ID NO: 14) on SKBR3 cells lysis (as HER2-positive breast cancer cells) in a 2D (two-dimensional) culture system comprising PBMCs and enriched NK cells (as exemplary CD16-positive cells), consistent with one or more exemplary embodiments of the present disclosure;

FIG. 3B illustrates a bar chart representing cytotoxic effect of an exemplary dsA7 (SEQ ID NO: 14) on MDAMB231 cells lysis in a 2D culture system comprising PBMCs and enriched NK cells, consistent with one or more exemplary embodiments of the present disclosure;

FIG. 4A illustrates a bar chart representing inhibitory effect of a fixed concentration (10 nM) of an exemplary dsA7 (SEQ ID NO: 14) on SKBR3 and MDAMB231 cells proliferation, compared to the inhibitory effects of Trastuzumab and an exemplary non-binding NC aptamer, consistent with one or more exemplary embodiments of the present disclosure;

FIG. 4B illustrates a bar chart representing inhibitory effect of different concentrations (0.25, 2, and 10 nM) of dsA7 (SEQ ID NO: 14) on SKBR3 cells proliferation compared to Trastuzumab, consistent with one or more exemplary embodiments of the present disclosure;

FIG. 5 illustrates graphs of flow cytometry binding analysis of exemplary anti-HER2 aptamers set forth in SEQ ID NOs: 1, 15 and 16 and an exemplary non-binding NC aptamer to SKBR3 and MDAMB231 cells, consistent with one or more exemplary embodiments of the present disclosure;

FIG. 6A illustrates graphs of flow cytometry binding analysis of an exemplary anti-CD16 aptamer set forth in SEQ ID NO: 3 and an exemplary non-binding NC aptamer to PBMCs, compared to an exemplary 3G8 antibody and an exemplary isotype control, consistent with one or more exemplary embodiments of the present disclosure;

FIG. 6B illustrates graphs of flow cytometry binding analysis of an exemplary anti-CD16 aptamer set forth in SEQ ID NO: 3 and an exemplary non-binding NC aptamer to enriched NK cells, compared to an exemplary 3G8 antibody and an exemplary isotype control, consistent with one or more exemplary embodiments of the present disclosure;

FIG. 7 shows bar graphs of normalized MFI values obtained from flow cytometry binding analysis of exemplary FITC-labeled dsA7 and dsA10 (SEQ ID NO: 14) to SKBR3 and enriched NK cells, consistent with one or more exemplary embodiments of the present disclosure; and

FIG. 8 illustrates lysis curve of SKBR3 cells in the presence of dsA7 (SEQ ID NO: 14) and in the presence of a combination of an exemplary 3G8 antibody with dsA7, consistent with one or more exemplary embodiments of the present disclosure.

### DETAILED DESCRIPTION

In the following detailed description, numerous specific details are set forth by way of examples to provide a thorough understanding of the relevant teachings related to the exemplary embodiments. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in one or more exemplary embodiments of one or more exemplary embodiments. For purposes of explanation, specific nomenclature is set forth to provide a thorough understanding of one or more exemplary embodiments. However, it will be apparent to one skilled in the art that these specific details are not required to practice the disclosed exemplary embodiments. Descriptions of specific exemplary embodiments are provided only as representative examples. Various modifications to the exemplary implementations will be plain to one skilled in the art, and the general principles defined herein may be applied to other implementations and applications without departing from the scope of one or more exemplary embodiments. Exemplary embodiments are not intended to be limited to the implementations shown, but is to be accorded the widest possible scope consistent with the principles and features disclosed herein.

Provided herein is an exemplary dual-specific aptamer, an exemplary method for producing an exemplary dual-specific aptamer, exemplary compositions comprising an exemplary dual-specific aptamer, and an exemplary method for cancer treatment using an exemplary dual-specific aptamer. “Dual-specific aptamer (dsA)” may refer to an aptamer/a nucleic acid molecule capable of binding, concurrently and specifically, to two markers/molecules/receptors, such as a protein marker on a cell surface. “Aptamer” may refer to single-stranded or double-stranded DNA (deoxyribonucleic acid) and/or RNA (ribonucleic acid) oligonucleotides capable of binding to different classes of molecules including, but not limited to, a protein, polypeptide, glycoprotein, lipid, glycopeptide, glycolipid, polysaccharide, and saccharide. Aptamers may be capable of forming secondary and/or tertiary and/or quaternary structures that may allow both specific and highly affine molecular interactions with various targets.

One or more exemplary embodiments is directed to an exemplary dual-specific aptamer comprising an exemplary anti-HER2 segment and an exemplary anti-CD16 segment. An exemplary dual-specific aptamer may specifically bind to a HER2 marker through an exemplary anti-HER2 segment and to an exemplary CD16 marker through an exemplary anti-CD16 segment. “HER2” (Human Epidermal growth factor Receptor 2)—also known as ErbB2 (erythroblastic oncogene B) and neu—may be a member of human EGFR (epidermal growth factor receptor) family. “CD16,” also known as FcγRIIIA, may refer to an exemplary cell-surface receptor on the surface of an exemplary cytotoxic effector cell capable of binding to a constant region (i.e., Fc) of antibodies and activating an exemplary antibody/aptamer-dependent cell-mediated cytotoxicity (ADCC) mechanism. CD16 receptor may include all FcγRIIIA isoforms and fragments and variants that may have CD16 biological activity. Exemplary isoforms of CD16 marker/receptor may include CD16a and CD160.

In one or more exemplary embodiments, an exemplary anti-HER2 segment of an exemplary dual-specific aptamer may be disposed at 5′ arm or 3′ arm of an exemplary dual-specific aptamer. In one or more exemplary embodiments, an exemplary anti-CD16 segment of an exemplary dual-specific aptamer may be disposed at 5′ arm or 3′ arm of an exemplary dual-specific aptamer. In an exemplary embodiment, an exemplary anti-HER2 segment may be disposed at 5′ arm of an exemplary dual-specific aptamer and an exemplary anti-CD16 segment may be disposed at 3′ arm of an exemplary dual-specific aptamer. An exemplary anti-HER2 segment may specifically bind to an exemplary HER2 marker on an exemplary HER2-positive cell. HER2-positive cancer cells, HER2-positive tumor cell(s), HER2-positive cancers, and/or HER2-positive tumors may refer to exemplary cancers and/or disorders and/or diseases with an increased expression (overexpression) of HER2 protein/gene/RNA transcript. In particular, any tumor/cancer cell exhibiting an upregulation of HER2 gene/transcript/protein may be identified as HER2-positive.

An exemplary anti-CD16 segment of an exemplary dual-specific aptamer may specifically bind to an exemplary CD16 marker on an exemplary cytotoxic effector cell. “Cytotoxic effector cell” or “effector cell” may refer to exemplary cells, generated through hematopoiesis, having cytolytical apoptosis-mediating or phagocytic properties. Cytotoxic effector cells may include T-lymphocytes (T-cells), monocytes, granulocytes, NK (natural killer) cells, mast cells, macrophages, and Langerhans cells. In an exemplary embodiment, an exemplary dual-specific aptamer, similar to exemplary commercial anti-HER2 antibodies, may be capable of binding specifically and simultaneously to both of exemplary HER2 and CD16 markers/receptors. In one or more exemplary embodiments, the bispecific binding of an exemplary dual-specific aptamer to an exemplary HER2 marker (on an exemplary HER2-positive cancer cell) and an exemplary CD16 marker (on an exemplary cytotoxic effector cell) may lead to activation of ADCC (antibody/aptamer-dependent cell-mediated cytotoxicity) against an exemplary HER2-positive cancer/tumor cell. “ADCC” may refer to an exemplary mechanism or phenomenon originating from interaction of Fcγ receptors (FcγR) (i.e., CD16) with specific binding sites in a constant (Fc)-region of exemplary antibodies and may play a pivotal role in treatment of various cancers. In particular, an exemplary ADCC mechanism may be activated upon interaction of an exemplary Fc-region of immunoglobulins with an exemplary FcγRIIIA/CD16 receptor expressed on the surface of an exemplary cytotoxic effector cell. Binding of an exemplary CD16 marker/receptor to an exemplary Fc-region may lead to secretion of granzyme and perforin that may enhance lysis of exemplary target cells. Binding of an exemplary CD16 marker/receptor to an exemplary Fc-region may also lead to the release of IFNγ (γ interferon) and recruitment of exemplary adaptive immune cells. During an exemplary ADCC mechanism, exemplary cytotoxic effector cells may induce exemplary death signals in exemplary target cells via death receptor/ligand ligation. Thus, in one or more exemplary embodiments, an exemplary dual-specific aptamer and/or exemplary compositions comprising an exemplary dual-specific aptamer may be effective for treating HER2-positive cancers (i.e., cancer cells that may overexpress HER2 marker/receptor). In one or more exemplary embodiments, an exemplary dual-specific aptamer and/or exemplary compositions comprising an exemplary dual-specific aptamer may be useful for treating HER2-positive cancers including, but not limited to, breast cancer, endometrial cancer, bladder carcinomas, gallbladder, anal cancer, colorectal cancer, uterine serous cancer (e.g., uterine papillary serous carcinoma), lung cancer (e.g., non-small-cell lung cancer), liver cancer, kidney cancer, gastroesophageal cancer, extrahepatic cholangio carcinomas, cervical cancer, uterine cancer, testicular cancer, ovarian cancer, pancreatic cancer, stomach cancer, etc. An exemplary dual-specific aptamer and/or exemplary compositions comprising an exemplary dual-specific aptamer may also be effective for treating any medical condition associated with HER2 marker/receptor dysregulation, e.g., overexpression. Exemplary medical conditions associated with HER2 marker/receptor dysregulation may include, but are not limited to, restenosis, arthritis, schizophrenia, and hyperproliferative diseases such as psoriasis.

In an exemplary embodiment, an exemplary dual-specific aptamer may comprise: i) an exemplary 5′ segment that may specifically bind to an exemplary HER2 marker on an exemplary HER2-positive cell (an exemplary anti-HER2 segment), ii) an exemplary 3′ segment that may specifically bind to an exemplary CD16 marker on an exemplary cytotoxic effector cell (an exemplary anti-CD16 segment), and iii) an exemplary linker adapted to attach an exemplary 5′ segment to an exemplary 3′ segment. Exemplary 5′ and 3′ segments may also be referred to as exemplary 5′ and 3′ arms, respectively. An exemplary 5′ arm may refer to an exemplary portion of an exemplary dual-specific aptamer that may be disposed at an exemplary 5′ end of an exemplary dual-specific aptamer or may constitute at least 10%, at least 20%, at least 30%, at least 40%, and/or at least 60% of nucleotides disposed near/next to an exemplary 5′ end of an exemplary dual-specific aptamer. An exemplary 3′ arm may refer to an exemplary portion of an exemplary dual-specific aptamer that may be disposed at an exemplary 3′ end of an exemplary dual-specific aptamer or may constitute at least 10%, at least 20%, at least 30%, at least 40%, and/or at least 60% of nucleotides disposed near/next to an exemplary 3′ end of an exemplary dual-specific aptamer.

In an exemplary embodiment, an exemplary anti-HER2 segment (an exemplary 5′ segment) of an exemplary dual-specific aptamer may specifically bind to an exemplary HER2 marker on an exemplary HER2-positive breast cancer cell. In an exemplary embodiment, an exemplary anti-CD16 segment) of an exemplary dual-specific aptamer may specifically bind to an exemplary CD16 marker on an exemplary cytotoxic effector cell. In one or more exemplary embodiments, binding of an exemplary dual-specific aptamer to an exemplary HER2-positive cancer cell (e.g., an exemplary HER2-positive breast cancer cell) and an exemplary cytotoxic effector cell may lead to degradation and/or internalization of exemplary HER2 markers/receptors, prevention of HER2 dimerization with exemplary EGFRs, cell cycle arrest, suppression of cell growth and/or proliferation (e.g., through inhibition of an exemplary MAPK (Mitogen-activated protein kinase) and PI3K (Phosphoinositide 3-kinases)/Akt signaling pathways), and cell lysis.

In an exemplary embodiment, an exemplary 5′ arm of an exemplary dual-specific aptamer may comprise an exemplary anti-HER2 segment and an exemplary 3′ arm of an exemplary dual-specific aptamer may comprise an exemplary anti-CD16 segment. In one or more exemplary embodiments, an exemplary anti-HER2 segment of an exemplary dual-specific aptamer may comprise an exemplary nucleic acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity to SEQ ID NO: 1. In an exemplary embodiment, an exemplary anti-HER2 segment may be at least 95 to 99.5% identical to SEQ ID NO: 1. In an exemplary embodiment, an exemplary anti-HER2 segment may be at least 98% identical to SEQ ID NO: 1. An exemplary anti-CD16 segment of an exemplary dual-specific aptamer may have an exemplary nucleic acid sequence selected from the group consisting of SEQ ID NO: 2 or SEQ ID NO: 3. In one or more exemplary embodiments, an exemplary anti-CD16 segment may comprise an exemplary nucleic acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity to any of SEQ ID NOs: 2 and/or 3. In an exemplary embodiment, an exemplary anti-CD16 segment may be at least 95 to 99.5% identical to any of SEQ ID NOs: 2 and/or 3. In an exemplary embodiment, an exemplary anti-CD16 segment may be at least 98% identical to any of SEQ ID NOs: 2 and/or 3. “Identity” may refer to a relationship between exemplary nucleic acid sequences of two or more exemplary polynucleotides that may be determined by comparing exemplary sequences of two or more exemplary polynucleotides. As appreciated by one skilled in the art, identity may also refer to an exemplary degree of relationship between the sequences calculated by determining number of matches between residues of two or more nucleic acid strings. % Identity applied to two or more exemplary polynucleotide sequences may refer to the percentage of residues (i.e., nucleic acid residues) in an exemplary candidate nucleic acid sequence that may be identical to the residues in an exemplary second nucleic acid sequence after aligning exemplary sequences and gap alignment, if necessary, to achieve a maximum percent identity. In one or more exemplary embodiments, exemplary variants of an exemplary polynucleotide (i.e., an exemplary reference polynucleotide) may have at least 40% to at least 99%, but less than 100%, sequence identity to an exemplary reference polynucleotide.

An exemplary dual-specific aptamer may further include an exemplary linker between an exemplary anti-HER2 segment (5′ arm) and an exemplary anti-CD16 segment (3′ arm). “Linker” may refer to exemplary nucleosidic and/or non-nucleosidic molecules or moieties that may physically couple/connect an exemplary anti-HER2 segment to an exemplary anti-CD16 segment. Exemplary nucleosidic linkers may not be limited to a precise sequence and length. An exemplary linker may connect one exemplary end of an exemplary anti-HER2 segment to one exemplary end of an exemplary anti-CD16 segment. Non-nucleosidic linkers may include, but are not limited to, glycol (e.g., polyethylene glycol), alkyl and amine linkers. In one or more exemplary embodiments, an exemplary linker of an exemplary dual-specific aptamer may be an exemplary nucleic acid linker including an exemplary single-stranded and/or an exemplary double-stranded nucleic acid linker. An exemplary nucleic acid linker may help an exemplary dual-specific aptamer to fold into an exemplary conformation/tertiary structure. In one or more exemplary embodiments, an exemplary nucleic acid linker may be an exemplary poly-Adenosine/Adenine (A) sequence ranging from 0 to 40 Adenine nucleotides. In an exemplary embodiment, an exemplary nucleic acid linker may have an exemplary nucleic acid sequence selected from the group consisting of SEQ ID NOs: 5 and 6, and/or an exemplary poly-A sequence consisting of 7 Adenosines/Adenines. An exemplary nucleic acid linker may have at least 50%, at least 60%, at least 70%, at least 80%, at least 90%, or 100% to SEQ ID NOs: 5 and 6, and/or an exemplary poly-A sequence consisting of 7 Adenosines/Adenines.

In an exemplary embodiment, an exemplary nucleic acid linker of an exemplary dual-specific aptamer may be double-stranded and may include an exemplary poly-A sequence ranging from 0 to 40 Adenine nucleotides that may be hybridized to an exemplary poly-Thymidine (T) sequence. An exemplary poly-A sequence of an exemplary double-stranded nucleic acid linker may have an exemplary nucleic acid sequence as set forth in SEQ ID NOs: 5 and 6, and/or an exemplary poly-A sequence consisting of 7 Adenosines/Adenines. In an exemplary embodiment, An exemplary poly-A sequence of an exemplary double-stranded nucleic acid linker may include an exemplary nucleic acid sequence with at least 50%, at least 60%, at least 70%, at least 80%, at least 90%, or 100% sequence identity to SEQ ID NOs: 5 and 6, and/or an exemplary poly-A sequence comprising 7 Adenosines/Adenines. In an exemplary embodiment, an exemplary single-stranded nucleic acid linker of an exemplary dual-specific aptamer may include an exemplary poly-A sequence as set forth in SEQ ID NO: 6. In an exemplary embodiment, an exemplary double-stranded nucleic acid linker of an exemplary dual-specific aptamer may include an exemplary poly-A sequence as set forth in SEQ ID NO: 6 that may be hybridized to an exemplary poly-T sequence set forth in SEQ ID NO: 7. Content and length of exemplary nucleic acid linkers may have a significant effect on binding affinity and binding durability of an exemplary dual-specific aptamer to both of exemplary HER2 and CD16 markers.

In one or more exemplary embodiments, an exemplary dual-specific aptamer may be designed based on different criteria including, but not limited to, position and content of each of exemplary anti-HER2 and anti-CD16 segments (being disposed either on 5′ or 3′ arms of an exemplary dual-specific aptamer), presence of an exemplary linker, type of an exemplary linker (being nucleosidic or non-nucleosidic), length of an exemplary nucleosidic linker, content of an exemplary nucleosidic linker, and being double-stranded or single-stranded.

In one or more exemplary embodiments, an exemplary dual-specific aptamer may have an exemplary nucleic acid sequence with at least 80%, 81%, 82%, 83%, 84%, 85%, 86%, 87%, 88%, 89%, 90%, 91%, 92%, 93%, 94%, 95%, 96%, 97%, 98%, 99%, or 99.5% sequence identity to any of SEQ ID NOs: 8 to 14. In an exemplary embodiment, an exemplary dual-specific aptamer may be at least 95 to 99.5% identical to any of SEQ ID NOs: 8 to 14. In an exemplary embodiment, an exemplary dual-specific aptamer may be at least 98% identical to any of SEQ ID NOs: 8 to 14. Table 1 below shows exemplary dual-specific aptamers (dsA1 to dsA10) that may have an exemplary anti-HER2 segment (SEQ ID NO: 1) and an exemplary anti-CD16 segment (any of SEQ ID NOs: 2 or 3), consistent with one or more exemplary embodiments. dsA(1 to 10) may stand for dual-specific aptamer (1 to 10); for example, dsA1 may refer to an exemplary dual-specific aptamer (1).

TABLE 1
Exemplary dual-specific aptamers (dsA1 to
dsA10) against exemplary HER2 and CD16
markers, consistent with one or more ex-
emplary embodiments of the present
disclosure.

| | 5′ | 3′ | | Sequence |
|---|---|---|---|---|
| | segment/ | segment/ | | ID |
| Title | aptamer | aptamer | Linker | number |
| dsA1 | Truncated | anti- | No | SEQ ID |
| | anti-CD16 | HER2 | linker | NO: 8 |
| | aptamer | aptamer | | |
| | (SEQ ID | (SEQ ID | | |
| | NO: 2) | NO: 1) | | |
| dsA2 | Anti-CD16 | Anti- | No | SEQ ID |
| | aptamer | HER2 | linker | NO: 9 |
| | (SEQ ID | aptamer | | |
| | NO: 2) | (SEQ ID | | |
| | | NO: 1) | | |
| dsA3 | Truncated | Anti- | 7 | SEQ ID |
| | anti-CD16 | HER2 | Adenosines/ | NO: 10 |
| | aptamer | aptamer | Adenines | |
| | (SEQ ID | (SEQ ID | | |
| | NO: 2) | NO: 1) | | |
| dsA4 | Anti-CD16 | Anti- | SEQ ID | SEQ ID |
| | aptamer | HER2 | NO: 5 | NO: 11 |
| | (SEQ ID | aptamer | | |
| | NO: 2) | (SEQ ID | | |
| | | NO: 1) | | |
| dsA5 | Truncated | Anti- | SEQ ID | SEQ ID |
| | anti-CD16 | HER2 | NO: 6 | NO: 12 |
| | aptamer | aptamer | | |
| | (SEQ ID | (SEQ ID | | |
| | NO: 2) | NO: 1) | | |
| dsA6 | Anti-HER2 | Truncated | SEQ ID | SEQ ID |
| | aptamer | anti-CD16 | NO: 6 | NO: 13 |
| | (SEQ ID | aptamer | | |
| | NO: 1) | (SEQ ID | | |
| | | NO: 2) | | |
| dsA7 | Anti-HER2 | anti-CD16 | SEQ ID | SEQ ID |
| | aptamer | aptamer | NO: 6 | NO: 14 |
| | (SEQ ID | (SEQ ID | | |
| | NO: 1) | NO: 2) | | |
| dsA8 | Truncated | Anti- | SEQ ID | SEQ ID |
| | anti-CD16 | HER2 | NO: 6 | NO: 12 |
| | aptamer | aptamer | (Double- | |
| | (SEQ ID | (SEQ ID | stranded) | |
| | NO: 2) | NO: 1) | | |
| dsA9 | Anti-HER2 | Truncated | SEQ ID | SEQ ID |
| | aptamer | anti-CD16 | NO: 6 | NO: 13 |
| | (SEQ ID | aptamer | (Double- | |
| | NO: 1) | (SEQ ID | stranded) | |
| | | NO: 2) | | |
| dsA10 | anti HER2 | anti-CD16 | SEQ ID | SEQ ID |
| | aptamer | aptamer | NO: 6 | NO: 14 |
| | (SEQ ID | (SEQ ID | (Double- | |
| | NO: 1) | NO: 3) | stranded) | |

In an exemplary embodiment, exemplary dual-specific aptamers referred to as dsA1 to dsA7 may have an exemplary single-stranded nucleic acid linker. Exemplary dual-specific aptamers referred to as dsA8, dsA9, and dsA10 may have an exemplary double-stranded nucleic acid linker. An exemplary double-stranded nucleic acid linker may include an exemplary poly-A sequence as set forth in SEQ ID NO: 6 which may be hybridized to an exemplary poly-T sequence set forth in SEQ ID NO: 7. In an exemplary embodiment, exemplary dual-specific aptamers of dsA1-5 and dsA8 may include: an exemplary anti-CD16 segment disposed at an exemplary 5′ arm of an exemplary dual-specific aptamer, and an exemplary anti-HER2 segment disposed at an exemplary 3′ arm of an exemplary dual-specific aptamer.

In one or more exemplary embodiments, exemplary dual-specific aptamers of dsA6, dsA7, dsA9, and dsA10 may include: an exemplary anti-HER2 segment disposed at an exemplary 5′ arm of an exemplary dual-specific aptamer, and an exemplary anti-CD16 segment disposed at an exemplary 3′ arm of an exemplary dual-specific aptamer. In an exemplary embodiment, an exemplary dual-specific aptamer may have an exemplary nucleic acid sequence set forth in SEQ ID NO: 14 and may include any of an exemplary dsA7 and an exemplary dsA10 set forth in Table 1. Exemplary dsA7 and exemplary dsA10 may have a similar nucleic acid sequence as set forth in SEQ ID NO: 14, but differing only in an exemplary nucleic acid linker segment. An exemplary dsA7 may have an exemplary single-stranded nucleic acid linker including A43 (Adenosine 43) to A62 of SEQ ID NO: 14 (an exemplary single-stranded nucleic acid linker may have an exemplary nucleic acid sequence set forth in SEQ ID NO: 6). An exemplary dsA10 may have an exemplary double-stranded nucleic acid linker that may be obtained from hybridization of A43 to A62 of SEQ ID NO: 14 to an exemplary poly-T sequence of SEQ ID NO: 7. In one or more exemplary embodiments, an exemplary dual-specific aptamer may comprise RNAs, DNAs, or a combination of DNAs and RNAs. In an exemplary embodiment, an exemplary dual-specific aptamer may comprise an exemplary modification or an exemplary segment (e.g., a sequence) that may lead to a desirable feature, such as increased stability, durability and affinity.

In an exemplary embodiment, an exemplary nucleic acid linker of dsA3, dsA4, dsA5, and dsA8 may link/attach 3′ end of an exemplary anti-CD16 segment to 5′ end of an exemplary anti-HER2 segment. In one or more exemplary embodiments, an exemplary nucleic acid linker of dsA6, dsA7, dsA9, and dsA10 may link/attach 3′ end of an exemplary anti-HER2 segment to 5′ end of an exemplary anti-CD16 segment.

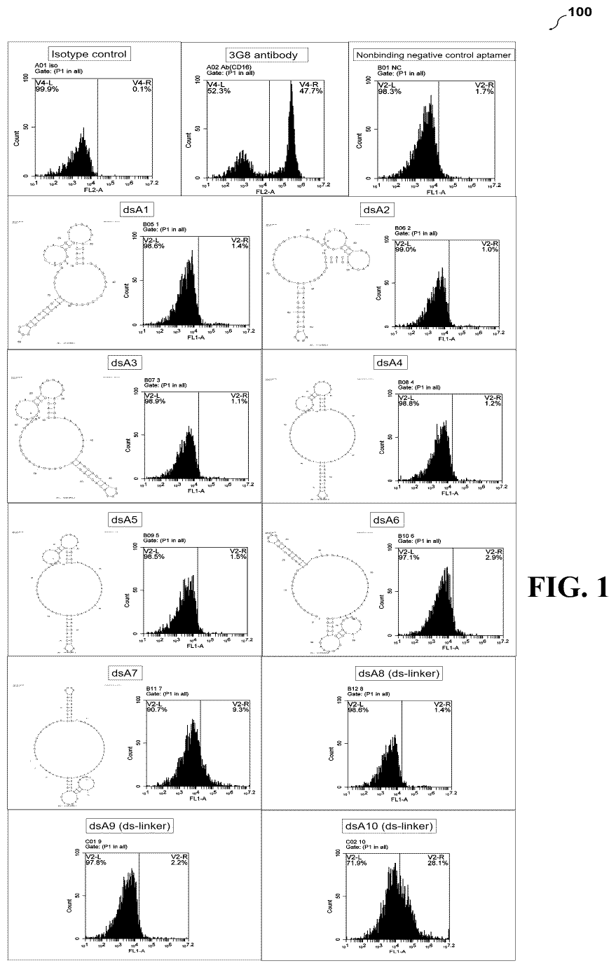

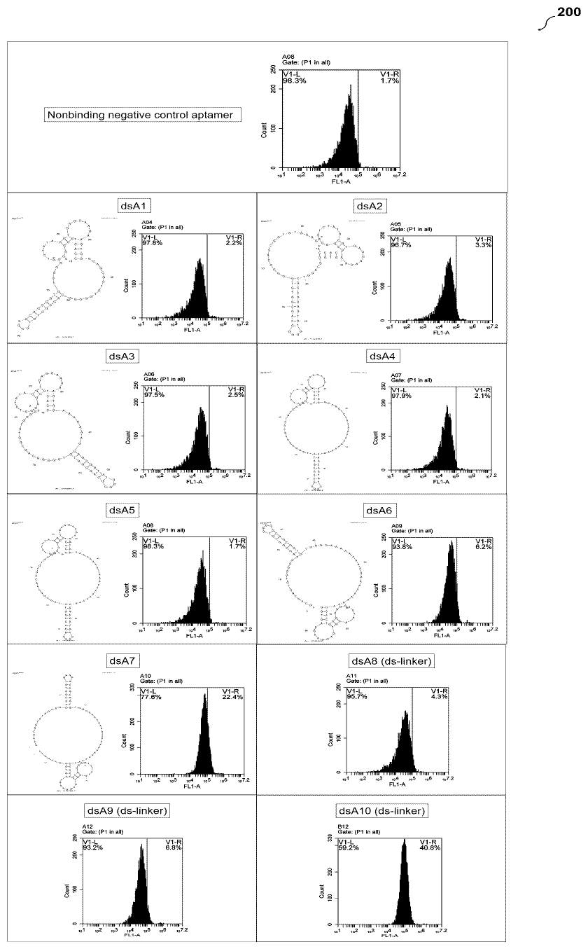

FIG. 1 illustrates predicted secondary structures 100 of exemplary dual-specific aptamers (dsA1 to 10) and their corresponding flow cytometry binding analysis after incubation with PBMCs (peripheral blood mononuclear cell) and enriched NK cells, consistent with one or more exemplary embodiments of the present disclosure. It may be appreciated by one skilled in the art that PBMCs and NK cell may have CD16 markers/receptors expressed on their surface. FIG. 2 illustrates predicted secondary structures 200 of exemplary dual-specific aptamers (dsA1 to 10) and their corresponding flow cytometry binding analysis after incubation with SKBR3 cells as HER2-positive breast cancer cells, consistent with one or more exemplary embodiments of the present disclosure. Referring to FIG. 1 and FIG. 2, exemplary dual-specific aptamers referred to as dsA7 and dsA10 may demonstrate a stronger and more durable binding to both of exemplary SKBR3 cells and exemplary PBMCs/NK cells, compared to dsA1-6, dsA8, and dsA9. In an exemplary embodiment, dsA7 and dsA10 may have a similar binding performance to binding performance of an exemplary anti-HER2 aptamer set forth in SEQ ID NO: 1 and an exemplary anti-CD16 aptamer of SEQ ID NO: 3 (i.e., exemplary monovalent aptamers) that both may have an optimal binding affinity to HER2 and CD16 markers, respectively (see FIGS. 1 and 2).

One or more exemplary embodiments may be directed to exemplary methods or techniques for producing/synthesizing an exemplary dual-specific aptamer, an exemplary anti-HER2 segment, and an exemplary anti-CD16 segment. In an exemplary embodiment, aptamers may be generated using an exemplary technique known as systematic evolution of ligands by exponential enrichment (SELEX). SELEX may comprise selection of aptamers on whole cells (cell SELEX) or on isolated recombinant protein (filter SELEX). In particular, SELEX may be an exemplary method for in vitro evolution of highly specific nucleic acid molecules (i.e., aptamers) against target molecules. Each SELEX-identified nucleic acid ligand (i.e., each aptamer) may be a specific ligand of an exemplary target compound or molecule. SELEX may be based on nucleic acids capability to form a variety of secondary and tertiary structures. It is to be understood that any of exemplary nucleic acid sequences disclosed in one or more exemplary embodiments may be prepared synthetically. Exemplary methods of synthetic oligonucleotide synthesis may include, but are not limited to solid-phase oligonucleotide synthesis, and liquid-phase oligonucleotide synthesis.

One or more exemplary embodiments may be directed to an exemplary method for treating an exemplary HER2-positive cancer in an exemplary subject in need thereof. An exemplary method may comprise administering an exemplary therapeutically effective amount of an exemplary dual-specific aptamer or an exemplary pharmaceutical composition comprising an exemplary dual-specific aptamer to an exemplary patient. “Therapeutically effective amount” may refer to an exemplary amount of an exemplary dual-specific aptamer or an exemplary pharmaceutical composition comprising an exemplary dual-specific aptamer that may treat an exemplary HER2-positive cancer in an exemplary subject including, but not limited to, tissue, system, animal, and human. Therapeutically effective amount may further refer to an exemplary amount that, in comparison to an exemplary reference subject who has not received an exemplary dual-specific aptamer, may decrease advancement of an exemplary disorder or disease, and may improve treatment, prevention, amelioration, or healing of an exemplary disorder, disease, or an exemplary side effect.

An exemplary method for treating an exemplary HER2-positive cancer may lead to lysis or growth-arrest of exemplary HER2-positive cancer cells. In one or more exemplary embodiments, an exemplary method may include administering an exemplary therapeutically effective amount of any of exemplary dual-specific aptamers including dsA1 to dsA10 (Table 1) to an exemplary patient. In an exemplary embodiment, an exemplary dual-specific aptamer may comprise an exemplary dsA7, an exemplary dsA10, or a combination thereof An exemplary dual-specific aptamer may comprise an exemplary dual-specific aptamer set forth in SEQ ID NO: 14 or an exemplary dual-specific aptamer having at least 60%, at least 70%, at least 80%, at least 90, at least 95%, or at least 99.5% sequence identity to SEQ ID NO: 14.

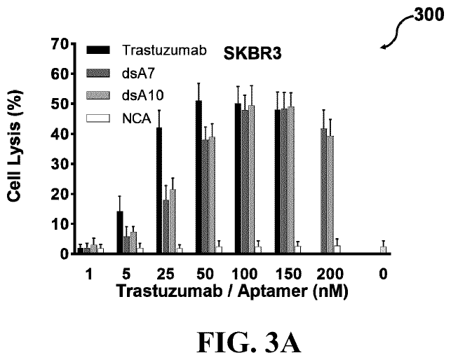

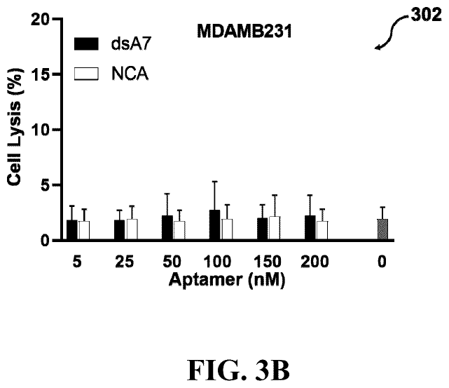

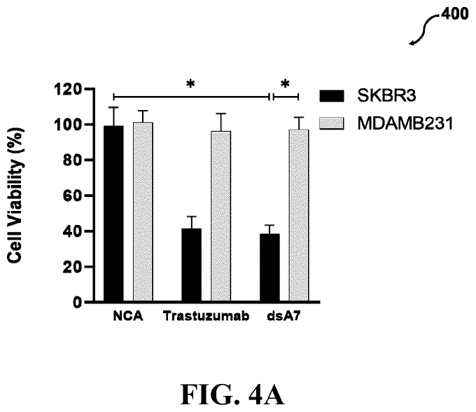

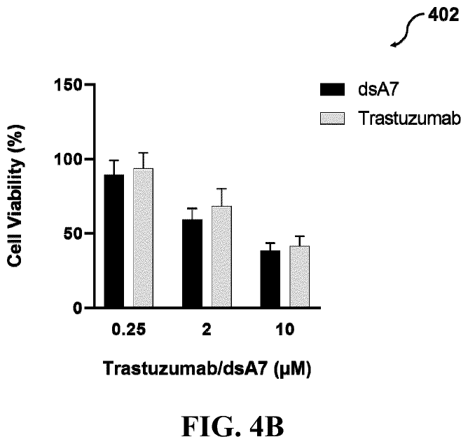

In an exemplary embodiment, an exemplary dual-specific aptamer may be used for treating HER2-positive breast cancer. FIG. 3A illustrates bar chart 300 representing cytotoxic effect of an exemplary dsA7 (SEQ ID NO: 14) and an exemplary dsA10 (SEQ ID NO: 14) on SKBR3 cells lysis (as HER2-positive breast cancer cells) in a 2D (two-dimensional) culture system comprising PBMCs and enriched NK cells (as exemplary CD16-positive cells), consistent with one or more exemplary embodiments of the present disclosure. FIG. 3B illustrates bar chart 302 representing cytotoxic effect of an exemplary dsA7 (SEQ ID NO: 14) on MDAMB231 cells lysis in a 2D culture system comprising PBMCs and enriched NK cells, consistent with one or more exemplary embodiments of the present disclosure. In further detail with respect to bar chart 300 and bar chart 302, both of an exemplary dsA7 and an exemplary dsA10 may have a similar performance as of an exemplary commercial anti-HER2 antibody (Trastuzumab) in mediating ADCC mechanism against exemplary SKBR3 cells (as exemplary HER2-positive breast cancer cells) and, in turn, SKBR3 cell lysis. FIG. 4A illustrates bar chart 400 representing inhibitory effect of a fixed concentration (10 nM) of an exemplary dsA7 (SEQ ID NO: 14) on SKBR3 and MDAMB231 cells proliferation, compared to the inhibitory effects of Trastuzumab and an exemplary non-binding NC aptamer, consistent with one or more exemplary embodiments of the present disclosure. FIG. 4B illustrates bar chart 402 representing inhibitory effect of different concentrations (0.25, 2, and 10 nM) of dsA7 (SEQ ID NO: 14) on SKBR3 cells proliferation compared to Trastuzumab, consistent with one or more exemplary embodiments of the present disclosure. Referring to FIGS. 3A-3B and FIGS. 4A-4B, an exemplary dsA7 aptamer may be capable of inhibiting SKBR3 cells proliferation, similar to anti-proliferation activity of Trastuzumab. In one or more exemplary embodiments, an exemplary effective amount of an exemplary dsA7 and an exemplary dsA10 may be effective for eliminating, suppressing, and/or inhibiting exemplary HER2-positive breast cancer cells.

### EXAMPLES

Hereinafter, one or more exemplary embodiments will be described in further detail with reference to examples. It will be obvious to a person having ordinary skill in the art that these examples may be for illustrative purposes only and are not to be interpreted to limit the scope of one or more exemplary embodiments.

### Example 1: Selection and Characterization of Exemplary Anti-HER2 and Anti-CD16 Aptamers to be Incorporated in an Exemplary Dual-Specific Aptamer

In this example, a number of exemplary anti-HER2 and anti-CD16 aptamers were selected as possible candidates to be incorporated in an exemplary dual-specific aptamer. Meanwhile, a random single-stranded DNA with a low free energy (dG) was obtained as an exemplary non-binding negative control (NC) aptamer using an exemplary online tool. Exemplary anti-HER2 aptamers may include SEQ ID NOs: 1, 15 and 16 and an exemplary anti-CD16 aptamer may include SEQ ID NO: 3. A truncated form of an exemplary anti-CD16 was also considered as a candidate to be used in designing an exemplary dual-specific aptamer; an exemplary nucleic acid sequence of an exemplary truncated anti-CD16 segment is set forth in SEQ ID NO: 2. General properties of exemplary anti-CD16 and anti-HER2 aptamers were analyzed using an exemplary computational molecular biology software that may be used for predicting secondary structure of single stranded nucleic acids. Table 2 below shows general properties including length, GC percent, and dG of exemplary secondary structures of exemplary anti-HER2 and anti-CD16 aptamers.

TABLE 2
General properties of exemplary anti-HER2
aptamers set forth in SEQ ID NOs: 1, 15
and 16, and an exemplary anti-CD16 aptamer
set forth in SEQ ID NO: 3, consistent with
one or more exemplary embodiments of the
present disclosure.

| Sequence | | Length | | dG of secondary |
|---|---|---|---|---|
| ID | | (nucleo- | GC | structure (Kcal/ |
| number | Target | tides) | % | mol at 37° C.) |
| SEQ ID | HER2 | 42 | 78.6 | −6.21 |
| NO: 1 | | | | |
| SEQ ID | HER2 | 86 | 43 | −0.06 |
| NO: 15 | | | | |
| SEQ ID | HER2 | 40 | 42.5 | −0.9 |
| NO: 16 | | | | |
| SEQ ID | CD16 | 41 | 61 | −2.01 |
| NO: 3 | | | | |

Exemplary aptamers set forth in SEQ ID NOs: 1, 3, 15 and 16, and an exemplary non-binding NC aptamer were conjugated to a biotin moiety and HPLC (high performance liquid chromatography)-purified. Exemplary anti-HER2 aptamers and an exemplary non-binding NC aptamer were 5′-biotinylated; an exemplary anti-CD16 aptamer was 3′-biotinylated.

Biotinylation was performed with the purpose of FITC (fluorescein isothiocyanate)-labeling and flow cytometry analysis. Binding activity of exemplary aptamers to their targets, i.e., CD16 and HER2, was evaluated on exemplary HER2-positive and CD16-positive cells. Therefore, SKBR3 (as HER2-positive breast cancer cells) and MDAMD231 cell lines (having low expression of HER2) were cultured in 10% FBS (fetal bovine serum)-supplemented DMEM (Dulbecco's Modified Eagle's medium) and were incubated at 37° C. with 5% CO2. On the other hand, peripheral blood mononuclear cells (PBMCs) were isolated from peripheral blood of healthy blood donors, using an exemplary protocol based on Ficoll density gradient centrifugation. To isolate and enrich Natural Killer (NK) cells (as CD16-expressing cells) from the isolated PBMCs (as CD16-expressing cells), a magnetic-activated cell sorting (MACS) NK cell isolation kit may be used.

In this example, binding properties of exemplary anti-HER2 aptamers (SEQ ID NOs: 1, 15 and 16) to HER2 and an exemplary anti-CD16 aptamer (SEQ ID NO: 3) to CD16 was evaluated by flow cytometry. To analyze binding performance of exemplary anti-HER2 and anti-CD16 aptamers, exemplary biotinylated anti-HER2 and anti-CD16 aptamers (SEQ ID NOs: 1, 3, 15 and 16) were initially conjugated with streptavidin (SA)-FITC. Next, SKBR3, MDAMB231, PBMCs and the isolated/enriched NK cells were detached, washed with PBS (phosphate buffer saline), and counted instantly using a Neobar lam; the ratio of dead to living cells was calculated for each cell type based on the enumeration results. SKBR3, MDAMB231, PBMCs were washed again with an exemplary washing buffer (PBS containing 0.1% BSA). Then, FITC-labeled aptamers (SEQ ID NOs: 1, 3, 15 and 16) and FITC-labeled nonbinding NC aptamer were prepared at an exemplary concentration of about 1000 nM in separate empty microtubes and heated at 95° C. for 5 minutes, followed by cooling at 25-37° C. for further refolding. SKBR3 and MDAMB231 cells were incubated with FITC-labeled anti-HER2 aptamers at 25° C. for 30 minutes. PBMCs and the enriched NK cells were incubated with FITC-labeled anti-CD16 aptamer at 25° C. for 30 minutes.

To determine purity of exemplary CD16-positive cells (i.e., PBMCs and enriched NK cells), a phycoerythrin (PE)-labeled anti-CD16 monoclonal antibody (3G8 antibody) was used as an exemplary flow cytometry control (reference). The incubated cells (samples) were analyzed by flow cytometry to evaluate binding properties of exemplary anti-HER2 and anti-CD16 aptamers. The mean fluorescence intensity (MFI) of SKBR3, MDAMB231, PBMCs, and NK cells bound to exemplary anti-HER2 and anti-CD16 aptamers was used as a reference to evaluate and interpret the specific binding performance of exemplary anti-HER2 and anti-CD16 aptamers.

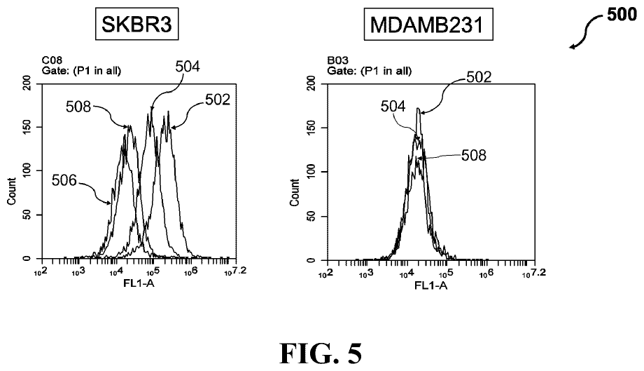

FIG. 5 illustrates graphs 500 of flow cytometry binding analysis of exemplary anti-HER2 aptamers set forth in SEQ ID NOs: 1, 15 and 16 (graph 502, graph 504 and graph 506, respectively) and an exemplary non-binding NC aptamer (graph 508) to SKBR3 and MDAMB231 cells, consistent with one or more exemplary embodiments of the present disclosure. In further detail with respect to graphs 500, an exemplary anti-HER2 aptamer set forth in SEQ ID NO: 1 (graph 502) demonstrated a robust binding to SKBR3 cells, compared to SEQ ID NO: 15 (graph 504) and SEQ ID NO: 16 (graph 506), while having no significant binding to MDAMB231 cells. Therefore, an exemplary anti-HER2 aptamer of SEQ ID NO: 1 was selected to be used for designing an exemplary dual-specific aptamer (i.e., dsAs set forth in Table 1).

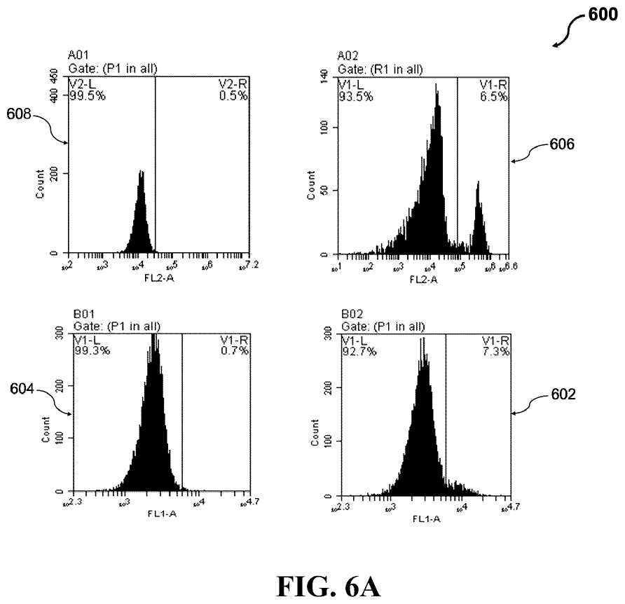

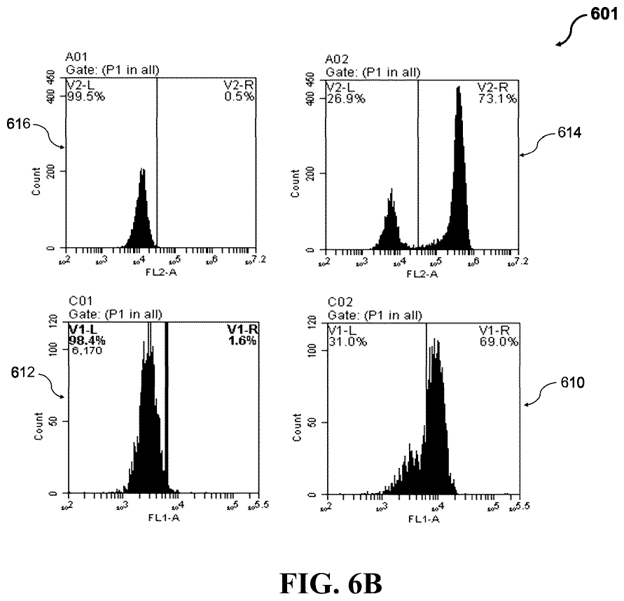

FIG. 6A illustrates graphs 600 of flow cytometry binding analysis of an exemplary anti-CD16 aptamer set forth in SEQ ID NO: 3 (graph 602) and an exemplary non-binding NC aptamer (graph 604) to PBMCs, compared to an exemplary 3G8 antibody (606) and an exemplary isotype control (graph 608), consistent with one or more exemplary embodiments of the present disclosure. FIG. 6B illustrates graphs 601 of flow cytometry binding analysis of an exemplary anti-CD16 aptamer set forth in SEQ ID NO: 3 (graph 610) and an exemplary non-binding NC aptamer (graph 612) to enriched NK cells, compared to an exemplary 3G8 antibody (graph 614) and an exemplary isotype control (graph 616), consistent with one or more exemplary embodiments of the present disclosure. In further detail with respect to graphs 600 and graphs 601, an exemplary anti-CD16 aptamer of SEQ ID NO: 3 demonstrated a strong and specific binding to PBMCs (graph 602) and enriched NK cells (graph 610). Meanwhile, an exemplary anti-CD16 aptamer of SEQ ID NO: 3 (graph 602 and graph 610) showed a binding performance similar to that of an exemplary 3G8 antibody (606 and 614) to both of PBMCs and enriched NK cells. An exemplary isotype control (i.e., an exemplary antibody used against an exemplary antigen that may not be found on exemplary sample cells) was used to ensure that the obtained MFIs are due to specific binding of an exemplary 3G8 antibody to exemplary target cells. The obtained results may approve the binding performance of an exemplary anti-HER2 aptamer of SEQ ID NO: 1 and an exemplary anti-CD16 aptamer of SEQ ID NO: 3, and may demonstrate that exemplary anti-HER2 and anti-CD16 aptamers may be useful for designing an exemplary dual-specific aptamer.

### Example 2: Designing a Plurality of Exemplary Dual-Specific Aptamers (dsA1-10) Against Both of HER2 and CD16 Markers/Receptors

In this example, a plurality of exemplary dual-specific aptamers were designed using an exemplary anti-HER2 aptamer of SEQ ID NO: 1 and an exemplary anti-CD16 aptamer of SEQ ID NO: 3. As discussed above, each of the plurality of exemplary dual-specific aptamers may include an exemplary anti-HER2 segment and an exemplary anti-CD16 segment, wherein an exemplary anti-HER2 and an exemplary anti-CD16 segments may be disposed 5′ or 3′ arms of an exemplary dual-specific aptamer. An exemplary dual-specific aptamer may be capable of binding, specifically and concurrently, to an exemplary HER2 marker (through an exemplary anti-HER2 segment) and an exemplary CD16 marker (through an exemplary anti-CD16 segment).

To design exemplary dual-specific aptamers with preserved fundamental binding properties and competent performance, ten different structures were designed and tested (i.e., dsA1-10 set forth in Table 1). Exemplary criteria for designing exemplary dual-specific aptamers may include, but is not limited to: i) truncation, orientation and position of exemplary anti-HER2 and anti-CD16 aptamers on an exemplary dual-specific aptamer, ii) the nucleotide content of an exemplary linker used for attaching exemplary anti-HER2 and anti-CD16 aptamers, and iii) length and number of strands of exemplary anti-HER2 and anti-CD16 aptamers (being either single- or double-stranded). Exemplary nucleic acid linkers may be single-stranded or double-stranded DNA/RNA with a wide variety of nucleotide contents and lengths, ranging from 7 to 20 nucleotides. Final length of an exemplary dual-specific aptamer may vary from 76 to 100 nucleotides.

In this example, each of the plurality of exemplary dual-specific aptamers (dsA1-10) were synthesized in biotinylated (5′ or 3′-biotinylated) and non-biotinylated forms; an exemplary biotinylated form may be used for FITC-labeling and flow cytometry analysis. An exemplary anti-CD16 segment of an exemplary dual-specific aptamer may be configured to trigger an ADCC (aptamer-dependent cell-mediated cytotoxicity) mechanism against exemplary HER2-positive cancer cells. Therefore, it may be predicted that an exemplary length of exemplary nucleic acid linkers may have a significant effect on activation of ADCC mechanism by an exemplary dual-specific aptamer. The optimal length of an exemplary linker may be determined based on the spanning distance between CDRs (complementarity-determining regions) and Fc-binding domain in an exemplary hinge region of exemplary anti-HER2 antibodies. An exemplary spanning distance has been estimated to be about 65 Å. Taking this exemplary distance into account, different linkers having exemplary lengths ranging from 0 to 128 Å may be considered for designing the plurality of exemplary dual-specific aptamers. Table 1 shown in the “DETAILED DESCRIPTION” section represents the plurality of exemplary designed and tested dual-specific aptamers (dsA1 to dsA10), consistent with one or more exemplary embodiments. The linker length of each respective dual-specific aptamer is set forth in Table 3 below.

TABLE 3
Linker properties of the designed exemplary
dual-specific aptamers (dsA1-10), consistent
with one or more exemplary embodiments of
the present disclosure.

| | | | Linker | Estimated |
|---|---|---|---|---|
| | Sequence | | length | Linker |
| | ID | | (nucleo- | length |
| Title | number | Linker | tides) | (Å) |
| dsA1 | SEQ ID | No linker | 0 | 0 |
| | NO: 8 | | | |
| dsA2 | SEQ ID | No linker | 0 | 0 |
| | NO: 9 | | | |
| dsA3 | SEQ ID | 7 | 7 | 44.8 |
| | NO: 10 | Adenosines/ | | |
| | | Adenines | | |
| | | (Single- | | |
| | | stranded) | | |
| dsA4 | SEQ ID | SEQ ID | 13 | 83.2 |
| | NO: 11 | NO: 5 | | |
| | | (Single- | | |
| | | stranded) | | |
| dsA5 | SEQ ID | SEQ ID | 20 | 128 |
| | NO: 12 | NO: 6 | | |
| | | (Single- | | |
| | | stranded) | | |
| dsA6 | SEQ ID | SEQ ID | 20 | 128 |
| | NO: 13 | NO: 6 | | |
| | | (Single- | | |
| | | stranded) | | |
| dsA7 | SEQ ID | SEQ ID | 20 | 128 |
| | NO: 14 | NO: 6 | | |
| | | (Single- | | |
| | | stranded) | | |
| dsA8 | SEQ ID | SEQ ID | 20 | 68 |
| | NO: 12 | NO: 6 | | |
| | | (Double- | | |
| | | stranded) | | |
| dsA9 | SEQ ID | SEQ ID | 20 | 68 |
| | NO: 13 | NO: 6 | | |
| | | (Double- | | |
| | | stranded) | | |
| dsA10 | SEQ ID | SEQ ID | 20 | 68 |
| | NO: 14 | NO: 6 | | |
| | | (Double- | | |
| | | stranded) | | |

In an exemplary embodiment, exemplary dual-specific aptamers (dsA1-10) set forth in Table 1 were incubated with an exemplary oligo-dT (deoxyThymidine/Thymine) sequence (an exemplary single-stranded sequence comprising 20 deoxyThymine nucleotides as set forth in SEQ ID NO: 7) at equal molar concentrations in order to be hybridized with an exemplary poly-Adenosine (A) linker of dsA5-10.

### Example 3: Evaluating Binding Properties of Exemplary Dual-Specific Aptamers (dsA1-10) to HER2 and CD16 Markers/Receptors

In this example, binding properties of dsA1-10 (set forth in Table 1) to HER2 and CD16 markers were evaluated by flow cytometry analysis. For this purpose, exemplary biotinylated forms of exemplary dual-specific aptamers set forth in Table 1 were conjugated with SA-FITC conjugates. With further reference to FIG. 1 and FIG. 2, FIG. 1 illustrates predicted secondary structures 100 of exemplary dual-specific aptamers (dsA1 to 10) and their corresponding flow cytometry binding analysis after incubation with PBMCs and enriched NK cells, consistent with one or more exemplary embodiments of the present disclosure. The flow cytometry binding analysis of an exemplary non-binding NC aptamer and an exemplary PE-labeled 3G8 antibody are also provided as controls/references in FIG. 1.

FIG. 2 illustrates predicted secondary structures 200 of exemplary dual-specific aptamers (dsA1 to 10) and their corresponding flow cytometry binding analysis after incubation with SKBR3 cells as HER2-positive breast cancer cells, consistent with one or more exemplary embodiments of the present disclosure. The cytometry binding analysis of an exemplary non-binding NC aptamer is also shown in FIG. 2. It is to be understood that the secondary structures demonstrated in FIG. 1 and FIG. 2 may be indefinite and may vary based on different biological and/or physiological conditions. For example, the exact secondary structure of exemplary dual-specific aptamers having double-stranded nucleic acid linkers (i.e., dsA8-10) may not be predictable.

As illustrated in FIG. 1 and FIG. 2, exemplary dual-specific aptamers referred to as dsA7 and dsA10 demonstrated a robust binding to both of SKBR3 cells (i.e., exemplary HER2 markers on SKBR3 cells surface) and NK cells or PBMCs, compared to the rest of dual-specific aptamers set forth in Table 1 (i.e., dsA1-6 and dsA8-9). Meanwhile, no significant unspecific binding was observed. As shown in FIGS. 1 and 2, dsA7 and dsA10 may have a binding performance similar the binding performance of exemplary anti-HER2 and anti-CD16 aptamers set forth in SEQ ID NOs: 1 and 3. dsA10 retained 90.5% and 93.3% of primary maximal binding affinity of exemplary anti-HER2 and anti-CD16 aptamers (SEQ ID NOs: 1 and 3) to SKBR3 and NK cells, respectively. On the other hand, dsA7 retained 48.5% and 39% of primary maximal binding affinity of exemplary anti-HER2 and anti-CD16 aptamers (SEQ ID NOs: 1 and 3) to SKBR3 and NK cells, respectively. As shown in FIG. 1 and FIG. 2, flow cytometry binding analysis of exemplary FITC-labeled dsA1-10 was compared to binding performance of an exemplary FITC-labeled non-binding NC aptamer and an exemplary isotype control. As shown in FIG. 1, flow cytometry binding analysis of exemplary FITC-labeled dsA1-10 was also compared to binding performance of an exemplary PE-labeled 3G8 antibody as an exemplary reference.

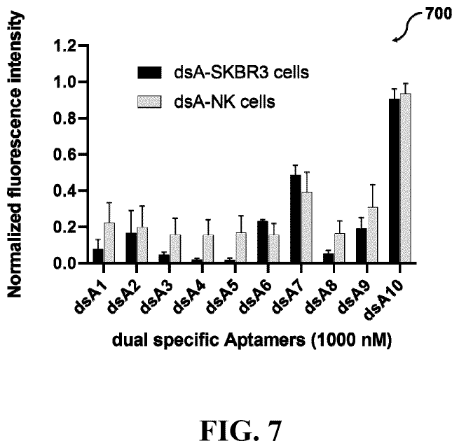

FIG. 7 shows bar graphs 700 of normalized MFI values obtained from flow cytometry binding analysis of exemplary FITC-labeled dsA7 and dsA10 (SEQ ID NO: 14) to SKBR3 and enriched NK cells, consistent with one or more exemplary embodiments of the present disclosure. To normalize exemplary MFI values of dsA7 and dsA10 based on their binding to each of SKBR3 and NK cells, the following formulae may be used:

$$Normalized MFI = \frac{MFI of a dual specific aptamer - MFI of the nonbinding NC aptamer}{MFI of the primary aptamer - MFI of the nonbinding NC aptamer} \qquad ( 1 )$$

An exemplary normalized MFI was calculated based on incubation of an exemplary fixed concentration (1000 nM) of dsA7 and dsA10 with SKBR3 and NK cells. Exemplary results shown in FIGS. 1-3 may further confirm that dsA7 and dsA10 may be suitable candidates for targeting exemplary HER2-overexpressing cancer cells (exemplary HER2-positive cancer cells) due to their specific and robust binding to both of HER2 and CD16 markers.

### Example 4: Evaluating Cytotoxic Effect of dsA7 and dsA10 by Performing an Exemplary ADCC Assay

In this example, cytotoxic effect of dsA7 and dsA10 was evaluated in a two-dimensional (2D) culture comprising breast cancer cells and enriched NK cells. An exemplary ADCC assay may be performed by measuring the amount of Lactate Dehydrogenase (LDH) released from damaged/lysed cells. Therefore, about 104 cells from each of SKBR3 and MDAMB231 cell lines, having at least 90% viability, were seeded and cultured per each well in a 96-well cell culture microplate using DMEM medium for SKBR3 and RPMI-1640 for MDAMB231. After 24 h of incubation at 37° C., culture mediums were removed and cells were washed once with PBS buffer. Then, 80 μL of DMEM and RPMI-1640 culture mediums, each containing different concentrations of dsA7 and dsA10 (ranging from 0 to 200 nM) were heated at 95° C. for 5 minutes. The heated culture mediums were cooled at 25° C., combined with 10 μL FBS, and added to each well. After 10 minutes of pre-incubation at 37° C. with 5% CO2, 10 μL of a culture medium containing 106 fresh PBMCs was added to each well to reach a final volume of 100 μL. The reaction plate was incubated at 37° C. with 5% CO2 for 4 hours, with slight shaking.

Specific cell lysis was estimated by measuring the amount of LDH released from damaged/lysed cells. For this purpose, cell-free supernatant of the above test reactions was separated from the incubated cells by centrifugation. Then, the amount of LDH in the separated cell-free supernatant was measured using an exemplary commercial cytotoxicity/LDH assay kit. The obtained signal from a medium-only sample was used as background and was subtracted from all measured signals. The following formulae was used to calculate percentage of target cells lysis:

$$\% specific lysis = \frac{sample lysis - spontaneous target cell lysis - spontaneous effector cell lysis}{maximum lysis - spontaneous target cell lysis} \times 100 \qquad ( 2 )$$

With further regards to FIG. 3A, FIG. 3A illustrates bar chart 300 representing cytotoxic effect of an exemplary dsA7 (SEQ ID NO: 14) and an exemplary dsA10 (SEQ ID NO: 14) on SKBR3 cells lysis (as HER2-positive breast cancer cells) in a 2D (two-dimensional) culture system comprising PBMCs and enriched NK cells (as exemplary CD16-positive cells), consistent with one or more exemplary embodiments of the present disclosure. The amount of cell lysis in the presence of dsA7 and dsA10 was compared to: i) the amount of SKBR3 cells lysis in the presence of an exemplary anti-HER2 monoclonal antibody (Trastuzumab), as an exemplary reference; and ii) the amount of cell lysis in the presence of an exemplary non-binding NC aptamer.

FIG. 3B illustrates bar chart 302 representing cytotoxic effect of an exemplary dsA7 (SEQ ID NO: 14) on MDAMB231 cells lysis in a 2D culture system comprising PBMCs and enriched NK cells, consistent with one or more exemplary embodiments of the present disclosure. In further detail with respect to bar chart 300 and bar chart 302, ADCC assay results of dsA7 and dsA10 was similar to ADCC assay of Trastuzumab antibody. No significant difference was observed between the ADCC assay results of dsA7 and dsA10. An optimal concentration of dsA7 and dsA10 led to at least 90% of the maximum cytotoxicity induced by Trastuzumab. As shown in bar chart 300 and bar chart 302, intensity of the induced cell cytotoxicity by dsA7 and dsA10 was completely dose-dependent (up to 150 nM). At higher concentrations of dsA7 and dsA10, ADCC-mediated cell lysis was significantly decreased.

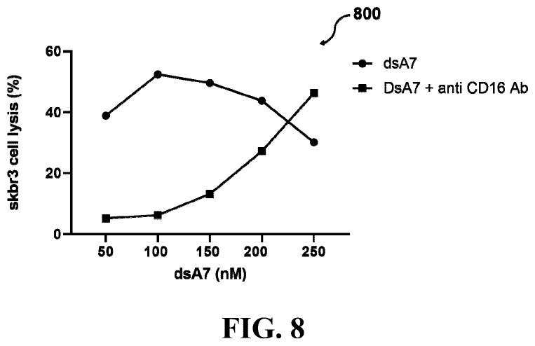

Direct role of CD16 marker as a lysis receptor may be demonstrated by blocking the CD16 receptors with an exemplary 3G8 antibody. Therefore, 150 nM of an exemplary 3G8 monoclonal antibody was added to each ADCC assay test reaction containing different concentrations of dsA7. The ADCC-mediated cell lysis was inhibited in the presence of a high concentration of an exemplary 3G8 antibody; this result may prove that dsA7 dual-specific aptamer may specifically bind from its anti-CD16 segment to CD16 marker and may lead to HER2-positive cells lysis by activating ADCC mechanism. FIG. 8 illustrates lysis curve 800 of SKBR3 cells in the presence of dsA7 (SEQ ID NO: 14) and in the presence of a combination of an exemplary 3G8 antibody with dsA7, consistent with one or more exemplary embodiments of the present disclosure.

### Example 5: Evaluating Ability of dsA7 to Inhibit Proliferation of HER2-Overexpressing Cells (SKBR3 Cells)

In this example, proliferation inhibition potential of dsA7 (SEQ ID NO: 14) was tested on SKBR3 and MDAMB231 cells, in vitro. For this purpose, 1000 cells from each of SKBR3 and MDAMB231 cell lines were seeded in each well of a 96-well plate and cultured with DMEM/F12 medium. After 24 h of incubation, DMEM/F12 medium was removed and 100 μL of DMEM/F12 medium containing 2.5% FBS—in two groups, one comprising different concentrations of dsA7 and the other comprising Trastuzumab—was added to each well. Both groups were refreshed every two days in the following 6 days. Finally, cell proliferation was measured using an MTS 43-(4,5-dimethylthiazol-2-yl)-5-(3-carboxymethoxyphenyl)-2-(4-sulfophenyl)-2H-tetrazolium)) proliferation assay kit. With further regards to FIG. 4A and FIG. 4B, FIG. 4A illustrates bar chart 400 representing inhibitory effect of a fixed concentration (10 nM) of an exemplary dsA7 (SEQ ID NO: 14) on SKBR3 and MDAMB231 cells proliferation, compared to the inhibitory effects of Trastuzumab and an exemplary non-binding NC aptamer, consistent with one or more exemplary embodiments of the present disclosure. As shown in bar chart 400, a 10 nM concentration of dsA7 significantly inhibited proliferation of SKBR3 cells as compared to the Trastuzumab-treated SKBR3 cells. MDAMB231 cells were not significantly affected by dsA7.

FIG. 4B illustrates bar chart 402 representing inhibitory effect of different concentrations (0.25, 2, and 10 nM) of dsA7 (SEQ ID NO: 14) on SKBR3 cells proliferation compared to Trastuzumab, consistent with one or more exemplary embodiments of the present disclosure. Referring to FIGS. 4A-B, bar chart 402 (that represents inhibitory effect of dsA7) was dose-dependent and no significant difference was observed as compared to Trastuzumab at exemplary concentrations shown in FIG. 4B. In further detail with respect to bar chart 400, “*” may refer to a P value<0.05, indicating that dsA7 may significantly inhibit proliferation of SKBR3 cells; however, dsA7 had no significant effect on MDAMB231 cells proliferation, in vitro. Meanwhile, bar chart 402 (that represents inhibitory effect of dsA7) was directly dose-dependent and was similar and comparable to bar chart 400 (that represents inhibitory effect of Trastuzumab).

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. An element proceeded by “a” or “an” does not, without further constraints, preclude the existence of additional identical elements in the process, method, article, or apparatus that comprises the element.

Unless otherwise stated, all measurements, values, ratings, positions, magnitudes, sizes, and other specifications that are set forth in this specification, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study, except where specific meanings have otherwise been set forth herein. Relational terms such as “first” and “second” and the like may be used solely to distinguish one entity or action from another without necessarily requiring or implying any actual such relationship or order between such entities or actions.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A dual-specific aptamer consisting of the nucleic acid sequence set forth in SEQ ID NO: 14, the dual-specific aptamer consisting of:
an anti-HER2 segment capable of binding to a HER2 marker, the anti-HER2 segment comprising nucleotide 1 to nucleotide 42 of SEQ ID NO: 14;
an anti-CD16 segment capable of binding to a CD16 marker, the anti-CD16 segment comprising nucleotide 63 to nucleotide 103 of SEQ ID NO: 14; and
a nucleic acid linker comprising nucleotide 43 to nucleotide 62 (N43-N62) of SEQ ID NO: 14, the nucleic acid linker selected from the group consisting of a double-stranded nucleic acid linker or a single-stranded nucleic acid linker, wherein the double-stranded nucleic acid linker comprises a poly-thymidine (T) sequence hybridized to the N43-N62 of SEQ ID NO: 14.

2. The dual-specific aptamer of claim 3, wherein the poly-T sequence comprises SEQ ID NO: 7.

3. The dual-specific aptamer of claim 1, wherein the anti-HER2 segment is capable of binding to the HER2 marker on a HER2-positive cell.

4. The dual-specific aptamer of claim 3, wherein the HER2-positive cell comprises a HER2-positive cancer cell.

5. The dual-specific aptamer of claim 1, wherein the anti-CD16 segment is capable of binding to the CD16 marker on a cytotoxic effector cell.
